(19) United States
(12) Patent Application Publication
(10) Pub. No.: US 2002/0131953 A1
(43) Pub. Date: Sep. 19, 2002

(54) In situ langerhans cell vaccine

(75) Inventors: Akira Takashima, Coppell, TX (US); Tadashi Kumamoto, Irving, TX (US)

Correspondence Address: BAKER & BOTTS, 30 ROCKEFELLER PLAZA, NEW YORK, NY, 10112

(73) Assignee: UT SOUTHWESTERN MEDICAL CENTER

(21) Appl. No.: 09/808,555

(22) Filed: Mar. 14, 2001

(51) Int. Cl. A61K 38/19; A61K 38/13; A61K 48/00; A61K 31/715

(52) U.S. Cl. 424/85.1; 424/423; 424/236.1; 514/44; 514/54; 514/9; 514/8

(57) ABSTRACT

A method for entrapping migratory antigen presenting cells (APCs) and particularly Langerhans cells (LCs,) in vivo is provided. The method entails creating an artificial gradient of APC-attracting chemotactic factor in the homing path of APCs in vivo. Also provided is a composition for entrapping APCs and particularly, migratory LCs. In addition, a method for loading APCs in situ with antigen is provided. The method comprises entrapping APCs in vivo and subsequently loading the APCs in situ with antigen. Correspondingly, a composition for loading APCs in situ is also provided. Further provided is a method for stimulating the migration of entrapped APCs to draining lymph nodes. The ability to stimulate the migration of entrapped APCs to draining lymph nodes is useful, inter alia, for regulating an immune response in a subject. In addition, an in situ APC-based vaccine is provided which does not require any time-consuming, costly ex vivo manipulations.

### SPECIFICATION

This invention was made with Government support under National Institutes of Health Grant #s RO1AR43777 and RO1AI43262. Therefore, the United States Government has certain rights in the invention.

### INTRODUCTION

The present invention relates to a method for regulating an immune response in a mammalian subject wherein an artificial gradient of a chemotactic factor is created in vivo and wherein said gradient allows for the transient entrapment of antigen presenting cells (APCs), such as Langerhans cells (LCs). The transient entrapment of APCs promotes the loading of the entrapped APCs with one or more immunoregulatory molecules, e.g. one or more antigens, one or more immunostimulatory molecules and/or one or more immunosuppressive molecules. After loading, the transiently entrapped APCs migrate to draining lymph nodes (DLN). The method of the present invention is useful for regulating an immune response in a mammalian subject, e.g., for treating or preventing cancer, treating or preventing infectious disease, treating or preventing autoimmune disease, treating or preventing allergic disorders, prolonging graft survival, treating or preventing graft versus host (GVH) disease and for inhibiting the inflammatory response. The present invention also relates to compositions for regulating an immune response in a mammalian subject.

### BACKGROUND OF INVENTION

A special subset of antigen presenting cells (APCs), called dendritic cells (DCs) plays a critical role in the initiation and regulation of immune responses and is instrumental in the induction of immunogenicity and the maintenance of tolerance (Banchereau and Steinman, Nature 392:245-252 (1998); Thomson and Lu, Transplantation 68:1-8 (1999)). Immunologically naive T cells can be activated most efficiently or even exclusively by DCs. See Banchereau & Steinman, 1998, Nature 392:245-252. T cell activation by DCs is necessary for the role of DCs in the immune response and can be defined by two distinct processes: (1) maturation, which involves the upregulation of major compatability complex (MHC) and costimulatory molecules, and (2) survival, which involves the rescue of DCs from immediate apoptosis after the withdrawal of growth factors. See Rescigno et al., J. Exp. Med. 188:2175-2180 (1998).

Mature DC expresses high levels of MHC class II and costimulatory molecules. In contrast, DCs with tolerogenic properties (such as immature DCs) express low levels of costimulatory molecules and induce antigen-specific hyporesponsiveness by triggering T cell apoptosis or clonal anergy. See Lu et al., Transplantation 60:1539-1545 (1995).

The immune system contains a system of DCs that is specialized to present antigens and initiate several T cell-dependent immune responses. DCs are distributed widely throughout the body in various tissues. DCs are found in nonlymphoid organs either close to body surfaces, such as in the skin and airways, or in interstitial regions of organs like heart and liver and can migrate via the blood and lymph to lymphoid organs (see Austyn et al., 1988, J. Exp. Med. 167:646, Larsen et al., 1990, J. Exp. Med. 171:307 and Austen and Larsen, 1990, Transpl. 49:1). There, antigens can be presented to T cells in the recirculating pool which, in turn, leads to an immune response (see Inaba et al., 1990, J. Exp. Med. 172:631).

The efficacy of DCs in delivering antigens in such a way that a strong immune response ensues i.e., “immunogenicity”, is widely acknowledged, but the use of these cells for delivering antigens is hampered by the fact that there are very few DCs in any given organ. In human blood, for example, only about 0.1% of the white cells are dendritic cells (Freudenthal et al., 1990, Proc. Natl. Acad. Sci. USA 87:7698). While DCs can process foreign antigens into peptides that must be recognized by immunologically active T cells (i.e., dendritic cells accomplish the phenomenon of “antigen presentation”), the low numbers of dendritic cells prohibits their use in identifying immunogenic peptides.

Injection of DCs pulsed with an idiotype protein of interest into mammals whereby immunity against lymphoma cells is induced is the subject of PCT patent application WO 91/13632. In addition, Francotte and Urbain, Proc. Nat'l. Acad. Sci. USA 82:8149 (1985) reported that mouse dendritic cells, pulsed in vitro with virus and injected back into mice, enhance the primary response and the secondary response to the virus. However Francotte, Urbain and patent application WO 91/13632 do not provide a practical method of using dendritic cells as an adjuvant to activate the immune response, because both of these methods depend on ex vivo techniques wherein dendritic cells are obtained from spleen, an impractical source of cells for most therapies or immunization procedures. In addition, neither Francotte nor Urbain provides a method to obtain dendritic cells in sufficient quantities to be clinically useful.

While DCs classically promote immune responses, they can be manipulated to induce antigen-specific hyporesponsiveness in vitro. The ability to manipulate the state of DC maturation in vitro has led to attempts to induce tolerance by administration of costimulatory molecule-deficient DCs in animal models of pancreatic islet cells or organ transplantation. See Fu et al., Transplantation 62:659-665 (1996); Rastelline et al., Transplantation 60:1366-1370 (1995); Lu et al., Transplantation 27:1808-1815 (1997); Gao et al., Immunology 98:159-170 (1999); Hirano et al., Transplant Proc. 32:260-264 (2000); Thomson and Lu, Transplantation 68:1-8 (1999). While these methods have had modest success, tolerance has not been achieved. This may be due to the late maturation/activation of DCs with upregulation of costimulatory molecules upon encountering a host microenvironment rich in pro-inflammatory mediators. The ability to manipulate the state of DC maturation may also be useful for the treatment of other diseases involving inflammatory events, such as autoimmune arthritis, asthma, septic shock, lung fibrosis, glomerulonephritis, atherosclerosis and AIDS.

U.S. Pat. No. 5,994,126 to Steinman et al. describes a method for in vitro proliferation of dendritic cell precursors and a method for producing mature dendritic cells from the precursors. This method requires ex vivo manipulation of a host's tissue source comprising DC precursors which are then cultured in vitro and matured in vitro and reintroduced back into the host.

In addition, anti-tumor immunity has been induced successfully in animal studies by the administration of tumor-associated antigens (TAA)-loaded autologous DCs. See Fong & Engleman, 2000, Annu. Rev. Immunol. 18:245-273.

Efforts have been made to develop genetic immunization with DCs infected with a viral vector expressing an antigen gene of interest. Tuting et al. (J. Gene Med. 1:400-406 (1999)) have shown that DCs infected with a recombinant adenovirus encoding tyrosinase-related protein-2 (TRP2) induces anti-melanoma immunity. In addition, DCs have also been used for vaccination against Epstein bar virus (EBV) by infecting DCs with an adenoviral vector encoding EBV antigens. Ranieri et al., J. Virol. 73:10416-10425 (1999).

Marked progress has been made with respect to the clinical application of DC-based vaccine strategies. First, short-term DC lines can be generated, as indicated above, by culturing human peripheral blood monocytes of CD34+ progenitors in the presence of selected cytokines. See U.S. Pat. No. 5,994,126; Caux et al., 1992, Nature 360:258-261; and Sallusto & Lanzavecchia, 1994, J. Exp. Med. 179:1109-1118. Second, DC loading with TAA has been achieved by adding crude tumor extracts, TAA proteins or TAA-derived peptides into DC cultures (see Grabbe et al., 1991, J. Immunol. 146:3656-3661; Paglia et al., 1996, J. Exp. Med. 183:317-322; and Mayordomo et al., 1995, Nature Med. 1:1297-1302) and also by introducing TAA-encoding DNA or RNA into DC (see Song et al., 1997, J. Exp. Med. 186:1247-1256; Specht et al., 1997, J. Exp. Med. 186:1213-1221; and Boczkowski et al., 1996, J. Exp. Med. 184:465-472).

Using such technologies, safety and efficacy of DC-based vaccines have been recently documented in patients with B cell lymphoma, malignant melanoma, renal cell carcinoma and prostate cancer. See Hsu et al., 1996, Nature Med. 2:52-58; Nestle et al., 1998, Nature Med. 4:328-332; Kugler et al., 2000, Nature Med. 6:332-336; and Lodge et al., 2000, Cancer Res. 60:829-833.

Genetic engineering of DCs to express immunosuppressive molecules has also been considered an attractive approach to alleviating foreign graft rejection and autoimmune disorders. See Lu et al., J. Leukoc. Biol. 66:293-296 (1999). Adenoviral delivery of cytotoxic T lymphocyte antigen 4-immunoglobulin (CTLA4Ig) into DCs has been shown to promote DCs in vitro tolerogenicity and survival in allogeneic recipients. Lu et al., Gene Ther. 6:554-563 (1999). In addition, delivery of transforming growth factor-β (TGF-β) using an adenoviral vector prevents the reduction of DCs generally seen with adenovirus infection and also increases the numbers and prolongs the survival of the infected DCs in the spleen of a host to whom the DCs are administered. Lee et al., Transplantation 66:1810-1817 (1998). Furthermore, U.S. Pat. No. 5,871,728 of Thomson et al., is directed to a method for enhancing tolerogenicity to a foreign graft in a host mammal comprising propagating immature DCs from a mammalian source, culturing the immature DCs in the presence of a cytokine and administering the propagated immature DCs to the host.

While modification of DCs may be an attractive approach to the therapy of cancer, infectious disease, foreign graft rejection, allergic disorders, GVH disease and autoimmune disorders, there are potential problems associated with such an approach. Although many DC-based vaccine protocols have been developed, the processes for developing them are time consuming and costly because they require ex vivo DC manipulation including isolation and expansion of DCs from individual hosts, manipulation of the maturational state of the DCs in culture, loading of the DCs in culture and administration of the loaded DCs back into the host. See Fong & Engleman, 2000, Annu. Rev. Immunol. 18:245-273. Such cumbersome, time-consuming and costly approaches limit the clinical application of DC vaccines.

The requirement of a series of ex vivo DC manipulation processes remains a major technical hurdle preventing DC-based vaccines from broader clinical applications. Several technologies have been developed primarily in the genetic vaccine field to bypass this hurdle. Utilizing mannose receptor 9 known to be expressed by DC as a targeting molecule, DC-targeted delivery of plasmid DNA has been achieved by using namman coated vehicles. See Sasaki et al., 1997, Eur. J. Immunol. 27:3121-3129. DC-targeted delivery of the gene product has been accomplished by designing plasmid DNA encoding TAA fused to granulocyte/macrophage colony-stimulating factor (GM-CSF) (known to bind DC). See Syrengelas et al., 1996, Nature Med. 2:1038-1041. One potential problem associated with delivering GM-CSF fused to TAA is that GM-CSF binds to other cell types which may lead to tolerance rather than immunity. In addition, Biragyn et al. (Nature Biotechnol. 17:253-258 (1999)) has fused TAA to monocyte chemotactic protein-3 (MCP-3), also known to bind DCs.

Based on the observation that tumor-specific CTL activities are inducible by intra-tumor injection of DC in the absence of TAA pulsing (see Lotze et al., Dendritic Cells: Biology and Clinical Applications, Lotze & Thomson (eds.), ;; 459-485 (Academic Press, San Diego, 1999)), Fushimi et al. (J. Clin. Invest. 105:1383-1393 (2000)), directly inoculated an adenoviral vector encoding MIP-3α (which is generally considered to attract immature DC) into tumors. However, if chemokine genes are injected directly without any pulsing with a particular antigen, fine antigen specificity may be lost which could lead to an autoimmune response.

PCT Publication No. WO 99/53912 describes a method for enhancing an immune response against an antigen by topical administration of an antigen or a portion thereof in conjunction with an enhancer of skin penetration and an induce or Langerhans cell migration. The antigen is preferably a peptide of 2 to 30 amino acids in length which is administered together with a means for enhancing penetration of the antigen. This method is limited because penetration of molecules into the skin is not very efficient. Correspondingly, the means for enhancing penetration of the antigen is necessary because the outermost layer of the skin, the stratum corneum, generally prevents penetration of molecules applied topically. In addition, it is very difficult to apply large molecules through the skin and therefore, the method is likely only effective with small peptides, as indicated (preferably the antigen is a peptide of 2 to 20 amino acids). Furthermore, since the Langerhans cells are only a small percentage of the epidermis (˜2%), for effective loading of a large population of Langerhans cells, a large area of the skin would probably need to be treated which would require large amounts of antigen.

There remains a need for an effective APC-based strategy which allows for the regulation of an immune response using loaded APCs which include any immunoregulatory molecule but which does not require inefficient, costly and time consuming ex vivo manipulations.

Langerhans cells (LCs) are specialized epidermal cells with dendritic morphology. GM-CSF and interleukin 1 mediate the maturation of murine epidermal LCs into potent immunostimulatory dendritic cells. Heufler et al., 1987, J. Exp. Med. 167:700-705). In addition, LCs in the epidermis which are specialized for antigen uptake and processing, are immature. Upon exposure to reactive haptens, LCs in the epidermis rapidly migrate to draining lymph nodes (DLNS) where they begin to exhibit mature features to develop into DCs. See Banchereau & Steinman, 1998, Nature 392:245-252. Chemokines and chemokine receptors are thought to control DC migration, which is essential for their maturation. See Cyster, 1999, J. Exp. Med. 189:447-450. Despite the above technologies, ex vivo Dc strategies have significant inadequacies that are overcome by the present invention.

### SUMMARY OF THE INVENTION

The present invention is directed to a method for entrapping Antigen Presenting Cells (APCs), including Dendritic Cells (DCs) and particularly, migratory Langerhans cells (LCs) in vivo. The method entails creating an artificial gradient of APC-attracting chemotactic factor in the homing path of APCs in vivo. The method for entrapping APCs in a subject comprises (a) administering a composition comprising one or more chemotactic factor(s) and (b) administering one or more APC stimulating factor(s) such that the APCs are transiently entrapped. In a preferred embodiment, the chemotactic factor(s) is administered subcutaneously and the APC stimulating factor(s) is administered topically. In a preferred embodiment, the composition comprising one or more chemotactic factor(s) also comprises a device.

The present invention is also directed to a composition for entrapping APCs, and particularly migratory LCs, which comprises a device and one or more chemotactic factor(s).

The present invention is further directed to a method for loading APCs in situ with one or more immunoregulatory molecules. As used herein, an immunoregulatory molecule may be inter alia an antigen, an immunostimulatory molecule or an immunosuppressive molecule. The method for loading APCs, and particularly LCs, a subject comprises (a) administering a composition comprising one or more chemotactic factor; (b) administering one or more APC stimulating factor; and (c) administering one or more immunoregulatory molecule wherein the APCs are loaded with the immunoregulatory molecule.

In addition, the present invention is directed to a method for stimulating the migration of APCs to draining lymph nodes comprising (a) administering to a subject a composition comprising a device and one or more chemotactic factor; and (b) administering one or more APC stimulating factor. The APCs migrate to the draining lymph nodes in a time delayed fashion. In a preferred embodiment, the composition comprising the chemotactic factor is administered subcutaneously and the APC stimulating factor is administered topically.

Furthermore, the present invention relates to a method for regulating an immune response which is useful for the treatment of, inter alia, cancer, infectious disease, allergic disorders, autoimmune disease and graft rejection. The method for regulating an immune response in a subject comprises (a) administering a composition comprising one or more chemotactic factor(s); (b) administering one or more APC stimulating factor(s); and (c) administering one or more immunoregulatory molecule(s) wherein APCs are loaded with the immunoregulatory molecule; and wherein the loaded APCs migrate to draining lymph nodes resulting in regulation of an immune response in the subject. In a preferred embodiment, the chemotactic factor and the immunoregulatory molecule are administered subcutaneously and the APC stimulating factor is administered topically. The method for regulating an immune response in a subject is useful to, inter alia, treat or prevent cancer, confer immunity against infectious disease, and achieve tolerogenicity for treating or preventing allergic disorders, autoimmune disease and allograft rejection.

Furthermore the present invention is directed to an APC-based in situ vaccine comprising (a) a composition comprising one or more chemotactic factor; (b) one or more immunoregulatory molecule; and (c) one or more APC stimulating factor.

### BRIEF DESCRIPTION OF THE DRAWINGS

The present invention may be better understood with reference to the attached drawings of which

FIG. 1 (a) is a graph showing the migration of mature spleen DCs (closed circles) and T cells (open circles) in upper chambers to the lower chambers in response to various concentrations of MIP-3β in lower chambers. (b) is a graph showing the migration of spleen DCs toward 10 ng/ml of MIP-3β in lower chambers in the presence of the indicated concentrations of MIP-3β in upper chambers. (c) is a graph showing the release of MIP-3β into media from EVA rods comprising MIP-3β at various time points. (d) is an immunoblot with anti-MIP-3β mAb showing the release of MIP-3β into media from EVA rods comprising MIP-3β after 3 days. (e) is a bar graph showing the biological activity of MIP-3β after release from EVA rods comprising MIP-3β.

FIG. 2 (a) is a bar graph showing LC densities at the site of MIP-3β rod and BSA rod (control) implantation in the skin of BALB/c mice by immunofluorescence staining with anti-IA and anti-DEC205 mAb. (b) is a bar graph showing IA⁺ LC densities after topical administration of DNFB onto implantation sites reflecting LC migration to DLN. (c) shows staining of cryostat sections using anti-IA mAb after MIP-3β rod implantation and DNFB application indicating that DNFB painting stimulates accumulation of IA⁺ in the vicinity of MIP-3β rods 24 hours after DNFB painting whereas DNFB did not stimulate accumulation of IA⁺ cells in the vicinity of BSA rods.

FIG. 3 (a & b) shows FACS profiles indicating the migration of LCs to DLN. FITC (a hapten with fluorescence) was applied over MIP-3β implantation sites. Recovery of IA⁺/FITC LCs was comparable for BSA rod implantation and no implantation groups indicating that local insertion of EVA polymer rods had no effect on LC homing whereas, MIP-3β rod implantation severely effected LC migration to DLN after 24 hours. (c) is a graph indicating that MIP-3β rods almost completely prevented LC homing to DLN in the first 24 hours, whereas the recovery of IA⁺/FITC LCs increased thereafter, reaching the same level as the BSA rod control group in 3 days indicating that MIP-3β rods entrap LCs transiently and they eventually home to DLN with ˜48 hour delay.

FIG. 4 shows the induction of tumor-specific CTL activities by in situ LC vaccine. (a) is a bar graph indicating CTL activities in spleen cells against OVA-transduced E.G7-OVA targets of mice receiving BSA rods, BSA/MIP-3β rods+BSA rods, BSA rods+OVA rods or MIP-3β/BSA rods+OVA rods in an OVA-transduced tumor line E.G7-OVA. (b) is a graphs indicating CTL activities in spleen cells against OVA-transduced E.G7-OVA targets of mice receiving coimplantation of MIP-3β rods+OVA rods (circles), BSA rods+OVA rods (squares) or MIP-3β rods on the back and OVA rods on the abdomen (triangles) in an OVA-transduced tumor line E.G7-OVA

FIG. 5 shows the prophylactic efficacies of in situ LC vaccine against tumor growth. Mice received co-implantation of the indicated rods on the abdomen, followed by DNFB application over the implantation sites and then they were challenged with E.G7-OVA tumor cells in the scapular region 5 days after rod implantation. (a) is a mouse model showing the reduction in size of tumors in mice receiving MIP-3β rods whereas mice which received BSA rods showed no reduction in tumor size. (b) is a bar graph indicating the reduction of tumor size in the mouse model.

FIG. 6 is a bar graph showing the therapeutic efficacies of in situ LC vaccine against tumor growth. Mice received in situ LC vaccines one day after tumor inoculation of E.G7-OVA tumor cells. Data shown are the tumor sizes 10 days after tumor inoculation.

### DETAILED DESCRIPTION

The present invention avoids the inadequacies of ex vivo DC-based vaccine strategies by providing an in situ method for entrapping migratory APCs, such as LCs, by creating an artificial gradient of APC-attracting chemotactic factor in the homing path of APCs. This entrapping method allows for the loading of APCs in situ. Such an in situ approach for loading APCs, such as LCs that ultimately mature into DCs, can circumvent the clinical complications of ex vivo DC-based strategies and can broaden the clinical applications of DC-based vaccines.

By combining APC entrapment technology and in situ APC loading technology, the present invention provides an APC-based immunoregulation format that requires no ex vivo manipulation. One key feature of the present invention is the ability to entrap APCs transiently without inhibiting their subsequent homing to DLN. In one embodiment, the entrapment is achieved by the administration of a composition comprising a device and one or more chemotactic factor, wherein the composition permits the controlled release of the chemotactic factor from the device. The entrapped APC can then be loaded in situ with one or more immunoregulatory molecule, such as an antigen which may be a tumor associated antigen (TAA) or an infectious disease-associated antigen. The entrapped APCs may also be loaded with immunosuppressive molecules that are capable of conferring tolerogenicity in a subject or immunostimulatory molecules. In addition, an antigen and an immunostimulatory molecule may be loaded into the DCs in situ. The present method is a unique method which allows for the administration of a protein, small molecules (such as synthetic peptides), DNA or RNA to achieve protective immunity to tumors and infectious disease, or to achieve tolerogenicity for treating allergic disorders, autoimmune disease and graft rejection

The present invention is based in part on the discovery that an artificial gradient of a chemotactic factor can be created in vivo in a mammalian subject. The artificial chemotactic factor gradient promotes the transient entrapment of antigen presenting cells (APCs) such as dendritic cells (DCs) and Langerhans cells (LCs). Therefore, the present invention provides a method for creating an artificial chemotactic factor gradient in vivo in a mammalian subject comprising administering to said subject a composition comprising one or more chemotactic factor(s). The present invention is correspondingly directed to a composition for creating a chemotactic factor gradient in a mammalian subject comprising one or more chemotactic factor(s).

As used herein, a chemotactic factor is any factor capable of attracting APCs, including, but not limited to, chemokines, nucleotides and neuropeptides. Nonlimiting examples of chemokines which are suitable in the present invention include MIP-1α, RANTES, MCP-3, MIP-5, MCPs, TARC, MDC, MIP-3α, IL-8, SDF-1, MIP-3β and SLC. Nonlimiting examples of nucleotides which are suitable in the present invention include ADP, UTP and UDP. In addition, neuropeptides such as, but not limited to, calcitonin-related gene protein (CGRP) and α-melanocyte-stimulating hormone (α-MSH) also may be used as the chemotactic factor of the present invention. See Dunzendorfer et al., 2001, Journal of Immunology 166:2167-2172. In a preferred embodiment, the chemotactic factor is MIP-3β.

The composition comprising one or more chemotactic factor(s) may further comprise a device. The chemotactic factor is incorporated into the device such that the chemotactic factor may be released from the composition comprising the device in a controlled manner. Nonlimiting examples of suitable devices include inter alia, nondegradable and biodegradable implant systems, implantable pump systems and atypical implantable pump systems.

Nonlimiting examples of nondegradable systems include nondegradable matrix systems such as polymeric matrix systems (monolitic systems), reservoir systems, beads systems, such as polymethylmethacrylate (PMMA), and polydimethylalloxane (PDMS) beads. Nonlimiting examples of biodegradable systems include reservoir systems and polymer monolitic systems, such as polyglycolic acid, polyactic acid, polyglycolic-lactic acid, polycaprolactone, ethylene-vinyl-acetate and actic acid/lysine. In addition, biodegradable copolymers with nondegradable coatings may be used, such as ethylene-vinyl-acetate/methacrylate. See Dash, 1998, Journal of Pharmacological and Toxicological Methods 40:1-12. In a preferred embodiment, the device is polyethylene-vinyl-acetate (EVA).

Nonlimiting examples of implantable pump systems include infusion pumps, peristaltic pumps, osmotic pumps, positive displacement pumps and controlled release micropumps. Nonlimiting examples of atypical implantable systems include ceramic composites, inorganic bone meal or ossograft, alminium calcium phosphorous oxide ceramics, hydroxyapetite ceramics, tricalcium phosphate and amino acid antibiotic composite ceramics, hydrogels, intraocular implants and transurethral systems. See Dash, 1998, Journal of Pharmacological and Toxicological Methods 40:1-12

In one embodiment, one or more chemotactic factor(s) is/are incorporated into the device. The chemotactic factor may be incorporated into the device by any means known in the art. For example, the chemotactic factor may be incorporated into the device by adding the chemotactic factor, preferably in a lyophilized dry state, to a device solution and subsequently lyophilizing the chemotactic factor/device mixture. The dry chemotactic factor/device mixture may then be melt-extruded into continuous rods. Example 1 describes one method for incorporating a chemotactic factor (e.g., MIP-3β) into a substrate (e.g., EVA). See Kim & Valentini, 1997, Biomaterials 18:1175-1184. Where the device is a pump, the chemotactic factor may be included in the pump in a suitable medium such that the chemotactic factor may be released from the pump in a controlled manner.

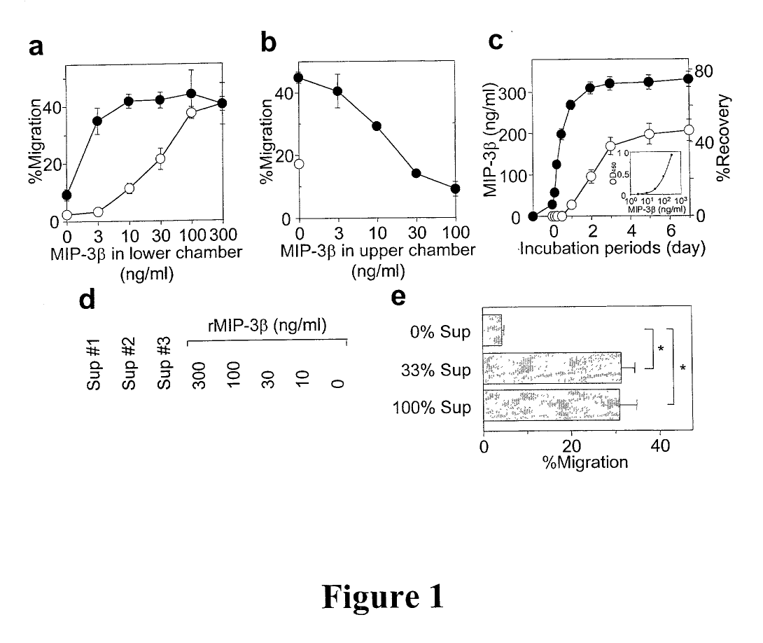

The chemotactic factor may be released from the device in a controlled manner. FIG. 1(c) and FIG. 1(d) show the release kinetics of MIP-3β from a composition comprising EVA rods. The released chemotactic factor is biologically active (see FIG. 1(d) and FIG. 1(e) and Example 3).

The present invention also relates to a composition for creating an artificial chemotactic factor gradient in a mammalian subject comprising one or more chemotactic factor and a device. In a preferred embodiment, the chemotactic factor is MIP-3β and the device is EVA. As noted above, the composition may be prepared according to Example 1 below. The chemotactic factor may be in the form of a protein, synthetic polypeptide, small molecule, ADP, UTP and UDP, gene, cDNA or RNA. Any molecule relating to a chemotactic factor may be incorporated into a composition comprising a device according to the present invention. Multiple chemotactic factors may be incorporated into the compositions of the present invention.

In addition, a nucleic acid molecule encoding a chemotactic factor, including a cDNA or RNA, may be included in a non-viral (plasmid) vector or viral vector by techniques well known in the art. For example, the chemotactic factor gene or corresponding cDNA or RNA may be incorporated into any suitable cloning or expression vector, operably linked to appropriate control elements (e.g. promoter elements, enhancer elements, ribosomal binding sites, polyadenylation sites, termination sites, etc.). Examples of such vectors include, but are not limited to, herpes simplex viral based vectors such as pHSV1 (Geller et al., 1990, Proc. Natl. Acad. Sci. U.S.A. 87:8950-8954); retroviral vectors such as MFG (Jaffee et al., 1993, Cancer Res. 53:2221-2226), and in particular Moloney retroviral vectors such as LN, LNSX, LNCX, LXSN (Miller and Rosman, 1989, Biotechniques 7:980-989); vaccinia viral vectors such as MVA (Sutter and Moss, 1992, Proc. Natl. Acad. Sci. U.S.A. 89:10847-10851); adenovirus vectors such as pJM17 (Ali et al., 1994, Gene Therapy 1:367-384; Berker, 1988, Biotechniques 6:616-624; Wand and Finer, 1996, Nature Medicine 2:714-716); adeno-associated virus vectors such as AAV/neo (Mura-Cacho et al., 1992, J. Immunother. 11:231-237); lentivirus vectors (Zufferey et al., 1997, Nature Biotechnology 15:871-875); and plasmid vectors such as pCDNA3 and pCDNA1 (InVitrogen), pET11a, pET3a, pET11d, pET3d, pET22d, pET12a and pET28a (Novagen); plasmid AH5 (which contains the SV40 origin and the adenovirus major late promoter), pRC/CMV (InVitrogen), pCMU II (Paabo et al., 1986, EMBO J. 5:1921-1927), pZipNeo SV (Cepko et al., 1984, Cell 37:1053-1062), pSRα (DNAX, Palo Alto, Calif.) and pBK-CMV, pSPTg.T2FpAXK and pSPTg.2FXK (Schaleger et al., 1997, Proc. Natl. Acad. Sci. U.S.A. 94:3058-3063).

APCs, including LCs, express chemokine receptors on their cell surface. These chemokine receptors bind to chemokines. As noted above, the artificial chemokine gradient allows for the transient entrapment of APCs in vivo. Therefore, the present invention also relates to a method for entrapping DCs and DC-related cells, such as LCs (collectively referred to herein as antigen presenting cells (APCs)), in situ comprising administering to a mammalian subject a composition comprising one or more chemotactic factor and administering one or more APC stimulating factor to the subject. In a preferred embodiment, the composition comprising the chemotactic factor is administered subcutaneously and the APC stimulating factor is administered topically.

As used herein, an APC stimulating factor is capable of stimulating APC migration and/or maturation. Nonlimiting examples of APC stimulating factors include reactive haptens; cytokines such as tumor necrosis factor-α (TNFα) and Interleukin 1 (IL-1); bacterial products such as lipopolysacharride (LPS) and lipoproteins (LPs) and ultraviolet radiation. See Smedt et al., 1996, J. Exp. Med. 184:1413-1424; Shornick et al., 1996, J. Exp. Med. 183:1427-1436; Cumberbatch & Kimber, 1994, Immunology 81:395-401; Cumberbatch & Kimber, 1992, Immunology 75:257-263; Stoltzner et al., 1999, Journal of Leukocyte Biology 60:462. As used herein, a reactive hapten is a molecule capable of stimulating APC migration and as acting as an antigen. Nonlimiting examples of reactive haptens include dinitroflourobenzene (DNFB), fluorescein isothiocyanate (FITC), FITC, oxazolone and urushiol. In one embodiment of the present invention, the reactive hapten may be DNFB or FITC.

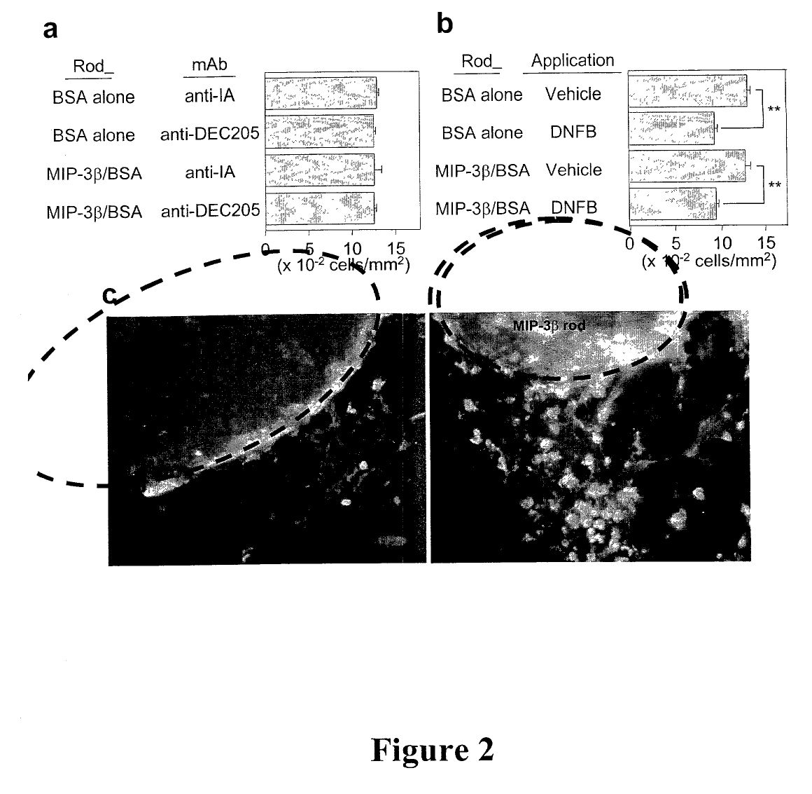

FIG. 1(a) and FIG. 1(b) show the ability of the compositions comprising a chemotactic factor of the present invention to facilitate the migration of spleen DCs towards the chemotactic factor (see Example 2). FIG. 2 (a) and FIG. 2 (b) illustrate the ability of the compositions of the present invention to effectuate the entrapment of LCs in vivo (see Example 4 below).

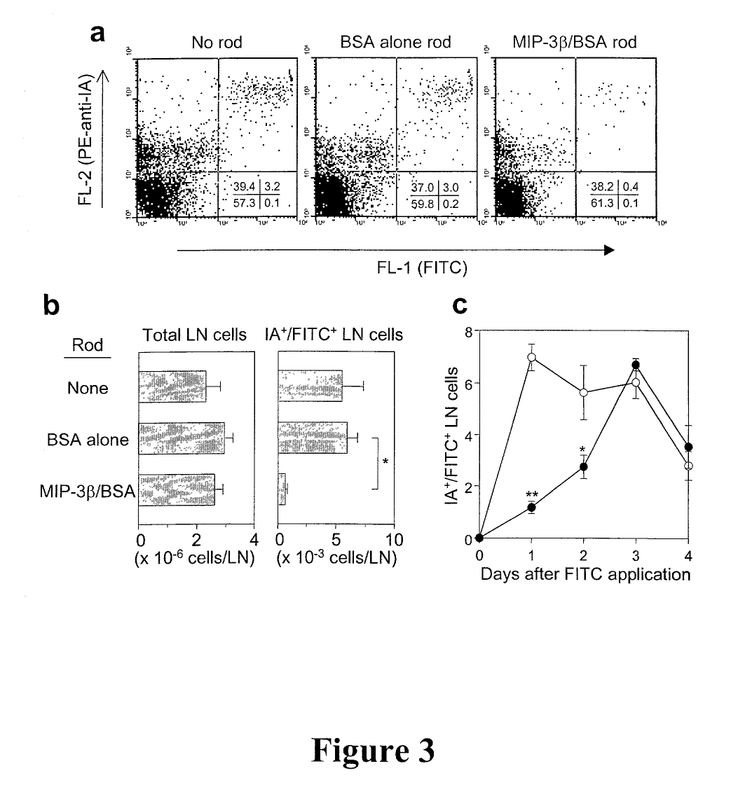

The entrapped APCs can effectively migrate to draining lymph nodes (DLN), a necessary step towards regulation of the immune response. See FIG. 3(a), FIG. 3(b) and FIG. 3(c) and Example 5 below. The migration of the APCs to DLN is time delayed, corresponding to the time in which the APCs are transiently entrapped.

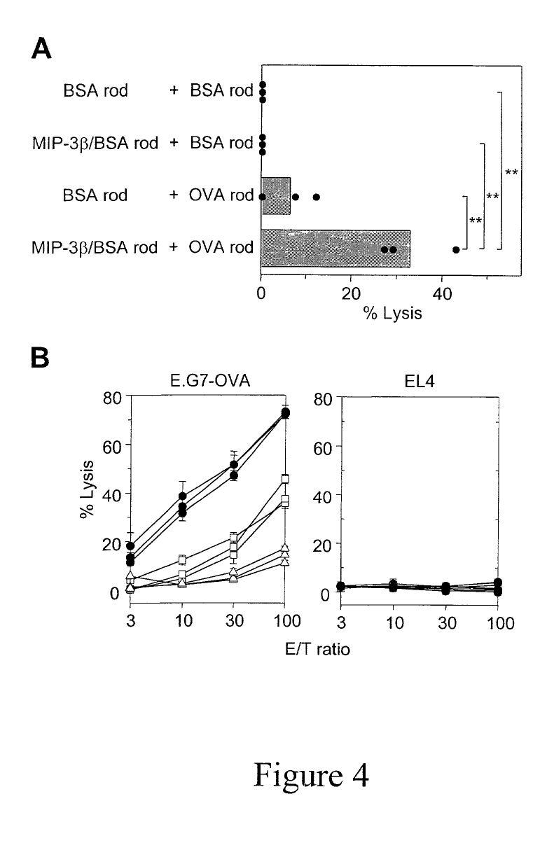

The entrapment of APCs allows for in situ loading of the APCs, e.g. LCs, with one or more immunoregulatory molecule, such as an antigen, an immunostimulatory molecule or an immunosuppressive molecule or combinations thereof. For example, the APCs may be loaded with a tumor associated antigen (TAA) wherein the APCs loaded with the TAA are capable of conferring protective immunity in the subject to tumors expressing the TAA. The transiently entrapped APCs loaded with one or more immunoregulatory molecule can migrate to DLN in a time-delayed manner. See FIGS. 3(a)-3(c) and Example 5 below. Thus, the present invention provides a method for loading APCs, and particularly LCs, in a subject comprising (a) administering a composition comprising one or more chemotactic factor; (b) administering one or more APC stimulating factor; and (c) administering one or more immunoregulatory molecule wherein the immunoregulatory molecule is loaded into the APC. In a preferred embodiment, the composition comprising one the chemotactic factor and the immunoregulatory molecule are administered subcutaneously and the APC stimulating factor is administered topically. FIGS. 4(a) through 4(c) and Example 6 demonstrate that TAA may effectively be loaded in vivo. The immunoregulatory molecule may be incorporated into a device alone or together with the chemotactic factor of the present invention.

As referred to herein, an immunoregulatory molecule can be any molecule, including, inter alia, an antigen, an immunostimulatory molecule or an immunoregulatory molecule or a combination thereof wherein said molecule(s) can regulate the immune response in a subject. The immunoregulatory molecule may be delivered as, inter alia, a protein, a synthetic polypeptide, a small molecule, a DNA, or an RNA. The immunoregulatory molecule may be included in a delivery vehicle such as a non-viral or viral vector, such as those described above, by techniques well known in the art. The immunoregulatory molecule can confer protective immunity in a subject to disease, including but not limited to, cancer and infectious disease (e.g. HIV). The immunoregulatory molecule may also be a molecule capable of enhancing tolerance in a mammalian subject to ameliorate inflammatory-related diseases, such as allergic disorders, autoimmune diseases, including but not limited to autoimmune arthritis, asthma, septic shock, lung fibrosis, glomerulonephritis, atherosclerosis, as well as AIDS. Other target disorders of the present invention include autoimmune skin diseases, such as lupus erythematosus, systemic sclerosis, dermatomyositis, bullous diseases, psoriasis, alopecia areata and vitiligo; allergic skin diseases, such as atopic dernatitis, allergic contact dermatitis and urticaria; bronchial asthma; allergic rhinitis; graft rejection; graft-versus-host disease (GVHD); inflammatory bowel disease; autoimmune neuro diseases; autoimmune endocrine diseases; autoimmune myopathy; solid cancers; leukemia; lymphoma and myeloma.

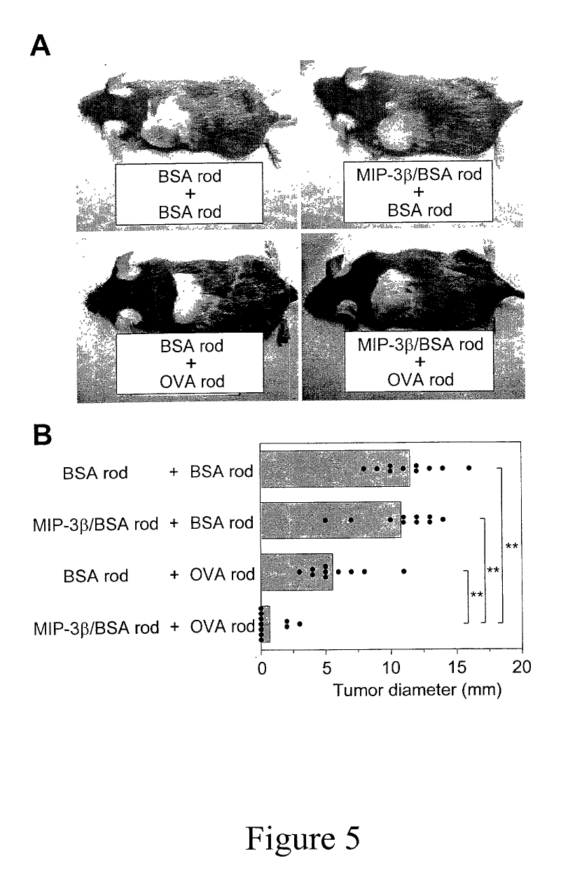

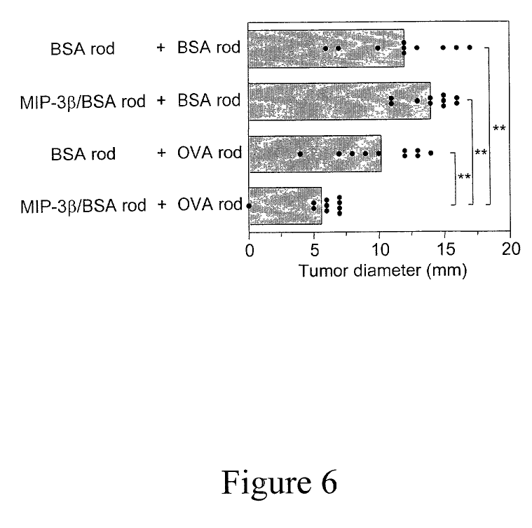

In one embodiment, the immunoregulatory molecule of the present invention may be, or may encode, inter alia, an antigen, such as a tumor-associated antigen (TAA), a self-antigen, an allogeneic antigen, a xenogeneic antigen or an infectious disease-associated antigen. Administration of a TAA or infectious disease-associated antigen may be prophylactic or therapeutic (see FIG. 5(a), FIG. 5(b) and FIG. 6). The present invention also provides for non-viral and viral vectors comprising one or more immunoregulatory molecule.

For example, the TAA may be a tumor specific antigen such as immunoglobulin idiotype (associated with non-Hodgkins' lymphoma), TCR (associated with T cell non-Hodgkin's lymphoma), mutant p21/ras (associated with pancreatic, colon and lung cancer), mutant p53 (associated with colorectal cancer, lung cancer, bladder cancer and head and neck cancer), p210/ber-abl fusion product (associated with chronic myelogenous leukemia and acute lymphoblastic leukemia). In addition, the TAA may be a developmental antigen such as MART-1/melan A (associated with melanoma), MAGE-1 and MAGE-3 (associated with melanoma, colorectal cancer, lung cancer and gastric cancer), GAGE family (associated with melanoma and telomerase (associated with may cancers). The TAA may also be a viral antigen such as human papilloma virus (associated with cervical cancer and penile cancer), and Epstein Bar virus (associated with Burkitt's lymphoma, nasopharyngeal carcinoma and post-transplant lymphopoliferative disorders). The TAA may further be a tissue-specific self antigen such as tyrosinase (associated with melanoma), gp100 (associated with melanoma), prostatic acid phosphatase (associated with prostate cancer), prostatic-specific antigen (associated with prostate cancer), prostate-specific membrane antigen (associated with prostate cancer), thyroglobulin (associated with thyroid cancer) and α-fetoprotein (associated with liver cancer). Additionally, the TAA may be an overexpressed self antigen such as Her-2/neu (associated with breast cancer and lung cancer), carcinoembryonic antigen (associated with colorectal cancer, lung cancer and breast cancer), Muc-1 (associated with colorectal cancer, pancreatic cancer, ovarian cancer and lung cancer) and telomerase (associated with numerous tumors, see Nair et al., 2000, Nature Med. 6:1011-1017). For a nonlimiting list of potential TAAs, see, e.g., Fong & Engleman, 2000, Annu. Rev. Immunol. 18:245-273.

Nonlimiting examples of allogenic antigens include class I MHC molecules, class II MHC molecules and non-classical MHC molecules or minor antigens. Allogeneic antigens may be useful for preventing or treating autoimmune disorders. In addition, the following nonlimiting examples of self-antigens are also useful for treating or preventing autoimmune disorders: Rh blood group antigens, such as I antigen, (associated with autoimmune hemolytic anemia), platelet integrins, such as GPIIb:IIIa, (associated with autoimmune thrombocytopenic purpura); non-collagenous domain of basement membrane collagen type IV (associated with Goodpasture's syndrome); epidermal cadherins (associated with Pemphigus vulgaris); stroptococcal cell-wall antigens (antibodies cross react with cardiac muscle; associated with acute rheumatic fever); rheumatoid factor IgG complexes (with or without hepatitis C antigens; associated with mixed essential cryoglobulinemia); DNA, histones, ribosomes, snRNP, scRNP (associated with systemic lupus erythematosus); pancreatic beta-cell antigen (associated with insulin-dependent diabetes mellitus); unknown synovial joint antigen (associated with rheumatoid arthritis); myelin basic protein, protolipid protein, and myelin oligodendrocyte glycoprotein (associated with experimental autoimmune encephalomyelitis (EAE) and multiple sclerosis). See Janeway C A, Travers P, Walport M, Capra J D, Autoimmunity: responses to self antigens, Immunobiology The Immune System in Health and Disease (Published by Current Biology Publications), 4th edition, 490-509, 1999.

The antigen may also be an infectious disease related antigen such as any antigenic molecule specific for a particular infectious disease, including infectious diseases caused by microorganisms.

In another embodiment, the immunoregulatory molecule of the present invention may be an immunosuppressive molecule including, but not limited to, cytokines such as IL-4, IL-10, IL16 and transforming growth factor-β (TGF-β); soluble co-stimulatory molecules such as cytotoxic T lymphocyte antigen 4-immunoglobulin (CTLA4-Ig), CD80-Ig, CD86-Ig and ICOS-Ig; neuropeptides such as CGRP and α-MSH; death ligands such as CD95 ligand and TRAIL; immunosuppressive chemicals such as corticosteroids, cyclosporin A, FK506 and rapamycin. According to the present invention, it is preferred that the immunosuppressive molecule be loaded into APCs together with an antigen to induce tolerogenicity in a subject.

In a further embodiment, the immunoregulatory molecule of the present invention may be an immunostimulatory molecule such as, but not limited to, cytokines such as GM-CSF, interferon-α, interferon-β, interferon-γ, IL-1, IL-2, IL-6, IL-7, IL-12, IL-15 and TNFα; co-stimulatory receptors such as CD40 ligand and inducible co-stimulatory protein (ICOS); and bacterial products such as lipopolysacharride (LPS) and lipoproteins (LP). The immunostimulatory molecule can boost the immunoregulatory response when given in combination with at least one other immunoregulatory molecule. In accordance with the present invention, the immunoregulatory molecule and the chemotactic factor may be the same. Therefore, the immune response may be regulated by adminstering one or more chemotactic factor in the absence of an immunoregulatory molecule if the chemotactic factor may also serve as an immunoregulatory molecule.

In accordance with the present invention, one or more immunoregulatory molecule(s) is/are loaded into the APCs to induce immunoregulation in an subject. For example, an immunosuppressive molecule may be loaded into APCs together with an antigen to induce tolerogenicity in a subject.

Accordingly, the present invention relates to a method for regulating an immune response in a subject comprising (a) administering a composition comprising one or more chemotactic factor(s); (b) administering one or more APC stimulating factor(s); and (c) administering one or more immunoregulatory molecule(s) wherein the immune response in the subject is regulated. In one embodiment, the immune response is stimulated (i.e. protective immunity is conferred), and in another embodiment, an immune response is inhibited (i.e. tolerogenicity is effectuated).

In addition, the present invention relates to an in situ APC-based vaccine comprising (a) a composition comprising one or more chemotactic factor(s); (b) one or more APC stimulating factor(s); and (c) one or more immunoregulatory molecule(s) wherein the vaccine regulates an immune response in a subject when administered thereto. The composition comprising the chemotactic factor and the immunoregulatory molecule may be administered subcutaneously and the APC stimulating factor may be administered topically. The method of the present invention may, for example, allow for the conferrence of protective immunity to a cancer in a subject. See FIGS. 4(a) through 4(c) and Example 7 below.

The invention is also directed to physiologic preparations of the compositions of the present invention which may be administered to a subject in a effective amount, i.e. an amount which can provide a chemotactic factor gradient, effect the transient entrapment of APCs, load entrapped APCs with immunoregulatory molecule(s) or regulate an immune response in a mammalian subject when administered thereto.

The compositions of the present invention may contain an effective dosage of chemotactic factor, immunoregulatory molecule or APC stimulating factor according to the present invention, together with a physiologically acceptable carrier and thus may be referred to as physiological compositions.

The invention is also directed to methods for regulating an immune response in a mammalian subject using the physiologic compositions of the invention. Such method comprises the administration of a physiological composition in an effective amount to a subject in need of immunoregulation. The dose of an effective amount may vary greatly depending on the molecule and the form of the molecule being administered, the condition, including size, age and weight of the subject and the disease to be treated. However, a determination of an effective amount may be readily determined by the skilled artisan.

The physiologic compositions may be administered parenterally by intramuscular or intravenous routes by aerosolization, subcutaneous administration, or oral, topical and intranasal administration. Preferably, the physiologic compositions comprising one or more chemotactic factor and/or one or more immunoregulatory molecule are administered subcutaneously and the APC stimulating factor is applied topically.

Generally, and by way of example only, for local administration, an effective amount of a composition comprising a chemotactic factor may be used at a concentration from about 100 ng/administration to about 3 mg/administration. Generally, and by way of example only, for local administration, an effective amount of antigen may be used at a concentration from about 100 ng/administration to about 30 mg/administration. For local administration, the immunostimulatory factor and the immunosuppressive factor may generally be used at a concentration from about 100 ng/administration to about 3 mg/administration. The devices of the present invention, when used in the form of a rod, for example, and for illustration purposes only, may be from about 0.1 mm to about 10 cm in diameter and from about 0.1 mm to about 20 cm in length. When used in a topical composition, the composition comprising APC stimulating factors can be used at a concentration from about 100 ng/ administration to about 10 mg/ administration.

The compositions of the present invention may be combined with other ingredients, such as carriers and/or adjuvants. There are no limitations on the nature of such other ingredients, except that they must be physiologically acceptable, efficacious for the intended administration and cannot degrade the active ingredients of the compositions. The compositions comprising APC stimulating factors can also be in the form of ointments or suspensions, and may be in combination with purified collagen. The compositions comprising APC stimulating factors also may be impregnated into transdermal patches, plasters and bandages, and be in a liquid or semi-liquid form.

The compositions of the present invention may also be systemically administered for promoting regulation of an immune response in a mammalian subject. When applied systemically, the compositions may be formulated as liquids, pills, tablets, lozenges or the like, for enteral administration, or in liquid form for parenteral injection. The compositions of the present invention intended for systemic administration may be combined with other ingredients such as carriers and/or adjuvants known to those skilled in the art. There are no limitations on the nature of such other ingredients, except that they must be physiologically acceptable, efficacious for their intended administration and cannot degrade the active ingredients of the compositions. Generally, when administered systemically, the chemotactic factor may be at a concentration from about 1 μg/kg to about 3 mg/kg body weight; the antigen may be from about 1 μg/kg to about 30 mg/kg body weight; the immunostimulatory factor may be from about 1 μg/kg to about 3 mg/kg body weight; and the immunosuppressive factor may be from about 1 μg/kg to about 3 mg/kg body weight.

The physiologic forms suitable for injection include sterile aqueous solutions or dispersions and sterile powders for the extemporaneous preparation of sterile injectable solutions or dispersions. In all cases the ultimate solution form must be sterile and fluid. Typical carriers known in the art include a solvent or dispersion medium containing, for example, water buffered aqueous solutions (i.e., biocompatible buffers), ethanol, polyol such as glycerol, propylene glycol, polyethylene glycol, suitable mixtures thereof, surfactants or vegetable oils.

Sterilization of the physiologic compositions can be accomplished by an art-recognized technique, including but not limited to, filtration or addition of antibacterial or antifungal agents, for example, paraben, chlorobutanol, phenol, sorbic acid or thimerosal. Further, isotonic agents such as sugars, for example, may be incorporated in the subject compositions. Production of sterile injectable solutions containing the subject compositions is accomplished by incorporating these compounds in the required amount in the appropriate solvent with various ingredients enumerated above, as required, followed by sterilization, preferably filter sterilization.

When the compositions of the invention are administered orally, the physiologic compositions thereof containing an effective dosage of the compositions may also contain an inert diluent, an assimilable, edible carrier and the like, be in hard or soft shell gelatin capsules, be compressed into tablets, or may be in an elixir, suspension, syrup, or the like. The subject compositions are thus compounded for convenient and effective administration in effective amounts with a suitable physiologically acceptable carrier in an immunoregulatory effective dosage.

The precise effective amount of the compositions to be used in accordance with this invention to regulate an immune response can be determined without undue experimentation by those skilled in the art who understand the nature of the immune response and the condition to be treated and/or prevented. The effective amount of the compositions that must be utilized can vary with the magnitude of the disease to be treated and/or prevented. The amount of the composition of the invention per unit volume of combined medication for administration may also be determined without undue experimentation by those skilled in the art. Systemic dosages also depend on the age, weight and conditions of the subject and on the administration route. As used herein, a physiologically acceptable carrier includes any and all solvents, dispersion media, coatings, and the like. The use of such media and agents are well known in the art.

Because the compositions of this invention may be designed to eliminate an ongoing infectious process, a continual application or periodic reapplication of the compositions may be indicated and preferred. The practice of the invention employs, unless otherwise indicated, conventional techniques of synthetic organic chemistry, protein chemistry, molecular biology, microbiology, recombinant DNA technology, and pharmacology, which are within the skill of the art. Such techniques are explained fully in the literature (See, e.g., Scopes, R. K. Protein Purification: Principles and Practices, 2nd edition, Springer-Verlag, 1987; Methods in Enzymology, S. Colwick and N. Kaplan, editors, Academic Press; Sambrook et al., Molecular Cloning: A Laboratory Manual, 2nd edition, Cold Spring Harbor Press, Cold Spring Harbor, N.Y.; Ausubel et al., Current Protocols in Molecular Biology, John Wiley and Sons, New York, 1995; Remington's Pharmaceutical Sciences, 17th ed., Mack Publishing Co., Easton, Pa., 1985).

Although only preferred embodiments of the invention are specifically described above, it will be appreciated that modifications and variations of the invention are possible without departing from the spirit and intended scope of the invention.

The following examples are provided to more clearly illustrate the aspects of the invention and are not intended to limit the scope of the invention.

### EXAMPLES

Example 1: Preparation of EVA polymer rods

All polymer rods were prepared in the Brown University laboratory of Dr. Robert F. Valentini. MIP-3β (900 ng/rod) was incorporated into the EVA polymer rods as described by Kim & Valentini, 1997, Biomaterials 18:1175-1184. Briefly, lyophilized MIP-3β was reconstituted in PBS, mixed with 1% BSA (1.8 mg/rod added as a carrier protein), snap frozen in liquid nitrogen, and re-lyophilized. EVA copolymer pellets (DuPont, Wilmington, Del.) were washed extensively and dissolved in methylene chloride to obtain a 1% solution (w/v). The lyophilized MIP-3β-BSA mixture was then added to the polymer solution with the ratio of 40:60 by weight, vortexed, snap frozen, and re-lyophilized. The resulting powder was melt-extruded at 50° C. into continuous polymer rods with a diameter of 0.7 mm. Rods were cut into 10 mm pieces and coated by dipping in a 5% EVA-methylene chloride solution. EVA rods containing BSA or OVA (1.8 mg/rod) were prepared as above in the absence of added MIP-3β. Release kinetics of MIP-3β and OVA were examined by ELISA and immunoblotting, respectively.

Example 2: In vitro migration of mature DCs in response to chemokine

Chemokines and chemokine receptors are thought to control DC migration. See Cyster, 1999, J. Exp. Med. 189:447-450. Several studies have shown that immature DC ordinarily express CCR6, whereas they acquire, upon maturation, CCR7 expression and a migratory property toward CCR7 ligands, i.e., MIP-3β and secondary lymphoid-tissue chemokine (SLC). See Dieu et al., 1998, J. Exp. Med. 188:373-386; and Saeki et al., 1999, J. Immunol. 162:2472-2475.

Several chemokines were tested in an in vitro transwell system for their ability to promote migration of mature DC, including MIP-1α, MIP-1γ, MCP-3, RANTES and MIP-3β. In vitro cell migration assays were performed by using 6.5 mm Transwell units (Costar, Cambridge, Mass.). Spleen DC or T cells prepared from BALB/c mice were placed in the upper chamber of the transwell system and chemokine (e.g., MIP-3β) was added to the lower chamber of the transwell system. After 2 hr incubation at 37° C., the migratory cells were harvested from the lower chambers, and the % DC (or T cell) migration was calculated by dividing the output CD11c⁺ (or CD3⁺) cells by the input CD11c⁺ (or CD3⁺) cells.

All the chemokines tested were able to promote spleen cell migration with the % specific migration being 8.2% for MIP-1α, 5.4% for MIP-1γ, 18.2% for MCP-3, 9.1% for RANTES and 13.2% for MIP-3β. Of the chemokines tested, MIP-3β was the most potent in promoting the migration of CD11c-positive splenic dendritic cell fractions. MIP-3β promoted migration of mature DCs at relatively low concentrations (3-10 ng/ml), whereas much higher concentrations were required for recruiting T cells (FIG. 1a; closed circles correspond to spleen DC and open circles correspond to T cells). FIG. 1b shows a chekerboard analysis which indicated the chemotactic nature of MIP-3β-promoted DC migration (closed circles indicate DC migration and open circles indicate baseline migration.

Example 3: Controlled release of active MIP-3β from EVA polymer rods

EVA polymer rods were formulated to release MIP-3β in a controlled fashion. EVA-MIP-3β rods were prepared as described above in Example 1. To determine whether MIP-3β was released from EVA polymer rods in a controlled manner and in an active form, MIP-3β-EVA rods were incubated in complete RPMI 1640 media (2 ml/rod) (Gibco-BRL, St. Louis, Mo.) for various time periods up to 6 days as indicated in FIG. 1c. FIG. 1c shows that MIP-3β was released from the rods into the media over the 6 day period (open circles. Closed circles represent data from MIP-3β rods which were first pre-incubated for 24 hours in a water phase (the water phase was complete RPMI for in vitro studies and PBS for in vivo studies) and cut into 4 short pieces before testing for MIP-3β release. An ELISA assay was used to determine MIP-3β concentrations in the media by using the standard curve shown in the insert in FIG. 1c. Data shown are the mean ±SD (n=3) of the cumulative concentrations and the % recovery. Presoaking the MIP-3β rods in a water phase and dissecting each rod into 4 shorter pieces further improved both release kinetics and recovery rates, producing >300 ng/ml MIP-3β release (>70% recovery) in the first 48 hr.

In addition, triplicate samples were collected on Day 3 and were examined by immunoblotting with anti-MIP-3β mAb, together with fresh recombinant MIP-3β serving as the standard. The immunoblot of FIG. 1d indicates that an immuno-reactive band of 9.4 kD corresponding to the size for intact MIP-3β was present in the media.

To determine whether the released MIP-3β was biologically active, the same samples as used above were tested at 33 or 100% (vol/vol) for their biological activities to promote T cell migration (FIG. 1e). DNFB-induced LC emigration was examined by counting the numbers of IA⁺ and DEC205⁺ epidermal cells after whole mount epidermal staining. See Mummert et al., 2000, J. Exp. Med. 192:769-779. The data in FIG. 1e show that the MIP-3β liberated from the EVA polymer exhibited significant biological activities in the chemotaxis assay.

Thus, MIP-3β can be liberated from the EVA polymer in a biochemically and functionally intact form.

Example 4: LC entrapment

After 24 hr pre-incubation in PBS at 37° C., a MIP-3β or BSA rod was cut into 4 short pieces (2.5 mm length) and implanted subcutaneously into a mouse with 200 μl of PBS using a 20G1/2 needle. At 24 hr post-implantation, 20 μl of 0.5% dinitroflourobenzene (DNFB) or 3% fluorescein isothiocyanate (FITC) was carefully applied over the implantation sites (10 mm-diameter circles marked with black ink immediately after implantation).

Subcutaneous implantation of EVA polymer rods containing MIP-3β (900 ng/rod/animal) did not affect the number of LC that remained in the overlaying epidermis (FIG. 2a) or cause significant accumulation of IA⁺ cells (IA⁺ serves as a marker for APCs) around the implanted rods at 24 hour pont-implantation. In an attempt to induce LC maturation and elevate MIP-3β responsiveness, DNFB was applied over the implantation sites. Significant (25-30%) reduction was observed in surface LC densities 24 hours after DNFB painting over BSA rod implantation sites, reflecting LC migration from the epidermis to DLN (FIG. 2b). DNFB application over MIP-3β rod implantation sites produced similar reduction in LC densities, indicating that MIP-3β rods did not promote or inhibit hapten-induced LC emigration from epidermis. Importantly, cryostat sections revealed marked accumulation of IA⁺ cells in the proximity of MIP-3β rods 24 hours after DNFB painting, whereas very few IA⁺ cells were observed around the BSA rods (FIG. 2c). Thus migratory LC can be entrapped by MIP-3β rod implantation, which creates an artificial chemokine gradient, followed by DNFB application which triggers LC migration and maturation.

Example 5: Homing of entrapped LC to DLN

To analyze LC homing to DLN, 3% FITC was applied over the rod-embedded sites and inguinal lymph node (LN) cells harvested at different time points were examined for IA⁺/FITC⁺ cell numbers by FACS. In some experiments, LN cell suspensions were enriched for DC by centrifugation through 14.5% metrizamide before FACS analysis and stained with PE-conjugated anti-IA mAb. See Love-Schimenti & Kripke, 1994, J. Immunol. 153:3450-3456, incorporated herein by reference.

To determine whether the LC which were entrapped by MIP-3β rods, as discussed above in Example 4, would eventually home to draining lymph nodes (DLN), FITC was applied over the implantation sites; IA⁺/FITC⁺ cells recovered from DLN should represent the LC that have migrated from FITC-painted skin sites because FITC acts not only as a reactive hapten but also as a fluorescence probe to label epidermal cells. See Love-Schimenti & Kripke, 1994, J. Immunol. 153:3450-3456. The recovery of IA⁺/FITC⁺ cells 24 hours after FITC painting was the comparable between the BSA rod implantation group and the no implantation group, indicating that the local insertion of EVA polymer rods had minimal effects on LC homing (FIG. 3a and FIG. 3b). In FIG. 3a and FIG. 3b, FITC was applied 24 hours after implantation f no rods, BSA rods or MIP-3β rods. Inguinal LN cells harvested 24 hours after FITC painting were enriched for DC by metrizamide treatment and stained with PE-conjugated anti-IA mAb. Data shown are two FACS profiles of representative samples (a) and the mean ±SEM (n=6) of the total LN cell numbers of IA⁺/FITC⁺ cells (b). Implantation of MIP-3β rods severely (80-90%) reduced LC migration to DLN after 24 hours.

The kinetics of LC homing after FITC was also examined. In FIG. 3c, FITC was applied over BSA rod (open circles) or MIP-3β rod implantation sites (closed circles) and LN cells harvested at the indicated time points were then examined for IA⁺/FITC⁺ LN cell numbers (mean ±SEM, n=4×10⁻⁴ cells/LN) in the absence of metrizamide treatment (*P<0.05, **P<0.01). MIP-3β rods prevented LC homing to DLN almost completely in the first 24 hours, whereas the recovery of IA⁺/FITC⁺ LN cells increased thereafter, reaching to the same level observed in the control BSA rod group 3 days after FITC painting. These results imply that LC are entrapped only transiently and that they eventually home to DLN with an approximate 48 hour delay.

Example 6: Loading of LC in situ

For in situ LC loading with antigen, a pre-soaked OVA rod (OVA was used as a model tumor associated antigen) containing 1.8 mg/rod/animal was cut into 4 pieces and co-implanted with a MIP-3β or BSA rod into an animal (C57BL/6 mice) and the implantation site was painted with DNFB 24 hr later. FIGS. 4a and 4b show induction of tumor-specific CTL activities an protective immunity by in situ LC loaded cells in three groups. The first group of C57BL/6 mice received co-implantation of MIP-3β rods+OVA rods on the abdomen followed by application of DNFB at the implantation site (circles), the second group received coimplantation of BSA rods+OVA rods on the abdomen followed by application of DNFB at the implantation site (squares) and the third group received implantation of MIP-3β rods on the back and OVA rods on the abdomen, followed by DNFB application at the site of implantation of the MIP-3β rods on the back. Spleen cells were harvested

Mice receiving MIP-3β rods+OVA rods developed potent CTL activities to lyse an OVA-transduced tumor cell line E.G7-OVA, but not the parental EL4 tumor cell line (FIGS. 4a & 4b). Co-implantation of BSA rods+OVA rods produced detectable CTL activities albeit in significantly lower magnitudes. No CTL activities were observed in control groups which received MIP-3β rods+BSA rods or BSA rods+BSA rods. Implantation of MIP-3β rods and OVA rods to distant locations (i.e. the back and abdomen) resulted in only marginal CTL activation, formally excluding the possibility that exogenously administered MIP-3β simply acts as a non-specific adjuvant.

Example 7: Prophylactic tumor protection in vivo

CTL and tumor growth assays: The OVA-transduced E.G7-OVA tumor line and the parental EL4 line have been previously described (see Porgador et al., 1996, J. Immunol. 156:2918-2926). CTL activities were measured by a standard ⁵¹Cr release assay after culturing spleen cells with γ-irradiated E.G7 cells for 5 days. See Porgador et al., 1996, J. Immunol. 156:2918-2926. Tumor cells (2×10⁷ tumor cells/animal) were subcutaneously injected into the scapular region of C57BL/6 mice and the tumor diameters were measured by a third experimenter blinded to sample identity.

The prophylactic efficacy of the method of the present invention was tested in an art recognized mouse tumor model. See Porgador et al., 1996, J. Immunol. 156:2918-2926. Five days after implantation with MIP-3β rods+OVA rods followed by DNFB application at the site of implantation, mice were inoculated with E.G7-OVA tumor cells. The control mice receiving BSA rods+BSA rods developed rapid tumor growth at the inoculation sites (FIGS. 5(a) & 5(b)). By contrast, mice which received MIP-3β rods+OVA rods showed almost full protection (FIGS. 5(a) & 5(b)). Consistent with the CTL data of Example 6 above, the second control group receiving BSA rods+OVA rods exhibited only partial protection, and no protection was observed in the third control group receiving MIP-3β rods+BSA rods. In three independent experiments, the in situ LC group receiving MIP-3β rods+OVA rods exhibited significantly (p<0.01) greater protection than did any of the three control groups, indicating reproducibility. Thus, anti-tumor protective immunity is readily inducible by loading the entrapped LC in situ with relevant TAA (in this case OVA).

With respect to short term safety, non of the >300 mice that received EVA polymer rods died during the experimental period or exhibited any apparent cutaneous manifestation at implantation sites.

Example 8: Therapeutic tumor protection in vivo:

The therapeutic efficacy of the method of the present invention was also tested in an art recognized mouse tumor model. Porgador et al., 1996, J. Immunol. 156:2918-2926. One days after inoculation with E.G7-OVA tumor cells, mice were implanted with MIP-3β rods+OVA rods followed by DNFB application at the site of implantation. The control mice receiving BSA rods+BSA rods once again developed rapid tumor growth at the inoculation sites with a marginal reduction (20-30%) in tumor growth for mice receiving BSA rods+OVA rods (FIG. 6). Mice which received MIP-3β rods+OVA rods showed 50-60% inhibition of tumor growth (FIG. 6). In two independent experiments, the in situ LC group receiving MIP-3β rods+OVA rods exhibited significantly (p<0.01) greater therapeutic efficacy than did any of the three control groups. These data indicate that tumor-specific protective immunity can be initiated by the methods and compositions of the present invention.

We claim:

1. A composition for providing an artificial chemotactic factor gradient in vivo comprising one or more chemotactic factor(s).

2. A composition for providing an artificial chemotactic factor gradient in vivo comprising one or more chemotactic factor(s) and a device.

3. A composition for providing an artificial chemokine gradient in vivo comprising one or more chemokine(s).

4. A composition for providing an artificial chemokine gradient in vivo comprising one or more chemokine(s) and a device.

5. A composition for providing an artificial chemokine gradient in vivo comprising one or more chemokine(s) and ethylene-vinyl-acetate.

6. A composition for entrapping antigen presenting cells in a subject comprising one or more chemotactic factor(s); and one or more APC stimulating factor(s).

7. A composition for entrapping antigen presenting cells in a subject comprising one or more chemokine(s) and one or more APC stimulating factor(s).

8. A composition for entrapping antigen presenting cells in a subject comprising one or more chemokine(s) and one or more reactive hapten(s).

9. A composition for loading antigen presenting cells in a subject comprising one or more chemotactic factor(s), one or more APC stimulating factor(s), and one or more immunoregulatory molecule wherein the immtnoregulatory molecule is loaded into the antigen presenting cells.

10. A composition for regulating an immune response in a subject comprising one or more chemotactic factor(s), one or more APC stimulating factor(s), and one or more immunoregulatory molecule(s).

11. The composition of claim 1 or 3, 6, 7, 8, 9 or 10 further comprising a device.

12. The composition of claim 6, 9 or 10 wherein the chemotactic factor is incorporated into a device.

13. The composition of claim 8 wherein the chemokine is incorporated into a device.

14. The composition of claim 9 or 10 wherein the immunoregulatory molecule is incorporated into a device.

15. The composition of claim 9 or 10 wherein the chemotactic factor and the immunoregulatory molecule are incorporated into a device.

16. The composition of claim 11, 12, 13, 14 or 15 wherein the device is selected from the group consisting of nondegradable implant systems, biodegradable implant systems, implantable pump systems and atypical implantable pump systems.

17. The composition of claim 11, 12, 13, 14 or 15 wherein the nondegradable implant system is selected from the group consisting of polymeric matrix systems (monolitic systems); reservoir systems; and bead systems, such as polymethylmethacrylate (PMMA) and polydimethylalloxane (PDMS) beads.

18. The composition of claim 11, 12, 13, 14 or 15 wherein the biodegradable implant system is selected from the group consisting of reservoir systems; polymer monolitic systems, such as polyglycolic acid, polyactic acid, polyglycolic-lactic acid, polycaprolactone, ethylene-vinyl-acetate and actic acid/lysine; biodegradable copolymers with nondegradable coatings, such as ethylene-vinyl-acetate/methacrylate.

19. The composition of claim 11, 12, 13, 14 or 15 wherein the implantable pump system is selected from the group consisting of infusion pumps, peristaltic pumps, osmotic pumps, positive displacement pumps and controlled release micropumps.

20. The composition of claim 11, 12, 13, 14 or 15 wherein the atypical implantable pump system is selected from the group consisting of ceramic composites, inorganic bone meal or ossograft, alminium calcium phosphorous oxide ceramics, hydroxyapetite ceramics, tricalcium phosphate and amino acid antibiotic composite ceramics, hydrogels, intraocular implants and transurethral systems.

21. The composition of claim 11, 12, 13, 14 or 15 wherein the device is ethylene-vinyl-acetate.

22. The composition of claim 1, 2, 6, 9 or 10 wherein the chemotactic factor is selected from the group consisting of chemokines, nucleotides and neuropeptides.

23. The composition of claim 3, 4, 5, 7, 8 or 22 wherein the chemokine is selected from the group consisting of MIP-1α, RANTES, MCP-3, MIP-5, MCPs, TARC, MDC, MIP-3α, IL-8, SDF-1, MIP-3β and SLC.

24. The composition of claim 22 wherein the nucleotide is selected from the group consisting of ADP, UTP and UDP.

25. The composition of claim 22 wherein the neuropeptide is selected from the group consisting of calcitonin-related gene protein and α-melanocyte-stimulating hormone.

26. The composition of claim 3, 4, 5, 7, 8 or 22 wherein the chemokine is MIP-3β.

27. The composition of claim 6, 7, 9 or 10 wherein the APC stimulating factor is selected from the group consisting of reactive haptens; cytokines, bacterial products, and ultraviolet radiation.

28. The composition of claim 8 or 27 wherein the reactive hapten is selected from the group consisting of dinitroflourobenzene, fluorescein isothiocyanate, oxazolone and urushiol.

29. The composition of claim 27 wherein the cytokine is selected from the group consisting of tumor necrosis factor-α and Interleukin 1.

30. The composition of claim 27 wherein the bacterial product is selected from the group consisting of lipopolysacharrides and lipoproteins.

31. The composition of claim 9 or 10 wherein the immunoregulatory molecule is selected from the group consisting of antigens, immunostimulatory molecules and immunosuppressive molecules.

32. The composition of claim 31 wherein the antigen is selected from the group consisting of tumor-associated antigens, self antigens, allogeneic antigens, xenogeneic antigens and infectious disease-associated antigens.

33. The composition of claim 32 wherein the tumor-associated antigen is selected from the group consisting of immunoglobulin idiotype, TCR, mutant p21/ras, mutant p53, p210/ber-abl fusion product, MART-1/melan A, MAGE-1, the GAGE family, human papilloma virus antigen, Epstein Bar virus antigen, tyrosinase, gp100, prostatic acid phosphatase, prostatic-specific antigen, prostate-specific membrane antigen, thyroglobulin, telomerase, Her-2/neu, carcinoembryonic antigen, and Muc-1.

34. The composition of claim 31 wherein the immunostimulatory molecule is selected from the group consisting of cytokines, co-stimulatory receptors, and bacterial products.

35. The composition of claim 34 wherein the cytokine is selected from the group consisting of GM-CSF, interferon-α, interferon-β, interferon-γ, IL-1, IL-2, IL-6, IL-7, IL-12, IL-15 and TNFA.

36. The composition of claim 34 wherein the co-stimulatory receptor is selected from the group consisting of CD40 ligand and inducible co-stimulatory protein.

37. The composition of claim 34 wherein the bacterial product is selected from the group consisting of lipopolysacharrides and lipoproteins.

38. The composition of claim 31 wherein the immunosuppressive molecule is selected from the group consisting of cytokines, soluble co-stimulatory molecules, neuropeptides, death ligands, and immunosuppressive chemicals.

39. The composition of claim 38 wherein the cytokine is selected from the group consisting of IL-4, IL-10, IL16 and transforming growth factor-β.

40. The composition of claim 38 wherein the soluble co-stimulatory molecule is selected from the group consisting of cytotoxic T lymphocyte antigen 4-immunoglobulin, CD80-Ig, CD86-Ig and ICOS-Ig.

41. The composition of claim 38 wherein the neuropeptide is selected from the group consisting of CGRP and α-MSH.

42. The composition of claim 38 wherein the death ligand is selected from the group consisting of CD95 ligand and TRAIL.

43. The composition of claim 38 wherein the immunosuppressive chemical is selected from the group consisting of corticosteroids, cyclosporin A, and FK506.

44. A method for providing an artificial chemotactic factor gradient in vivo comprising one or more chemotactic factor(s).

45. A method for providing an artificial chemotactic factor gradient in vivo comprising one or more chemotactic factor(s) and a device.

46. A method for providing an artificial chemokine gradient in vivo comprising one or more chemokine(s).

47. A method for providing an artificial chemokine gradient in vivo comprising one or more chemokine(s) and a device.

48. A method for providing an artificial chemokine gradient in vivo comprising one or more chemokine(s) and ethylene-vinyl-acetate.

49. A method for entrapping antigen presenting cells in a subject comprising one or more chemotactic factor(s); and one or more APC stimulating factor(s).

50. A method for entrapping antigen presenting cells in a subject comprising one or more chemokine(s) and one or more APC stimulating factor(s).

51. A method for entrapping antigen presenting cells in a subject comprising one or more chemokine(s) and one or more reactive hapten(s).

52. A method for loading antigen presenting cells in a subject comprising one or more chemotactic factor(s), one or more APC stimulating factor(s), and one or more immunoregulatory molecule(s) wherein the immunoregulatory molecule is loaded into the antigen presenting cells.

53. A method for regulating an immune response in a subject comprising one or more chemotactic factor(s), one or more APC stimulating factor(s), and one or more immunoregulatory molecule(s).

54. The method of claim 44, 46, 49, 50, 51, 52 or 53 further comprising a device.

55. The method of claim 49, 52, 53 wherein the chemotactic factor is incorporated into a device.

56. The method of claim 51 wherein the chemokine is incorporated into a device.

57. The method of claim 52 or 53 wherein the immunoregulatory molecule is incorporated into a device.

58. The composition of claim 52 or 53 wherein the chemotactic factor and the immunoregulatory molecule are incorporated into a device.

59. The method of claim 54, 55, 56, 57 or 58 wherein the device is selected from the group consisting of nondegradable implant systems, biodegradable implant systems, implantable pump systems and atypical implantable pump systems.

60. The method of claim 54, 55, 56, 57 or 58 wherein the nondegradable implant system is selected from the group consisting of polymeric matrix systems (monolitic systems); reservoir systems; and bead systems, such as polymethylmethacrylate (PMMA) and polydimethylalloxane (PDMS) beads.

61. The method of claim 54, 55, 56, 57 or 58 wherein the biodegradable implant system is selected from the group consisting of reservoir systems; polymer monolitic systems, such as polyglycolic acid, polyactic acid, polyglycolic-lactic acid, polycaprolactone, ethylene-vinyl-acetate and actic acid/lysine; biodegradable copolymers with nondegradable coatings, such as ethylene-vinyl-acetate/methacrylate.

62. The method of claim 54, 55, 56, 57 or 58 wherein the implantable pump system is selected from the group consisting of infusion pumps, peristaltic pumps, osmotic pumps, positive displacement pumps and controlled release micropumps.

63. The method of claim 54, 55, 56, 57 or 58 wherein the atypical implantable pump system is selected from the group consisting of ceramic composites, inorganic bone meal or ossograft, alminium calcium phosphorous oxide ceramics, hydroxyapetite ceramics, tricalcium phosphate and amino acid antibiotic composite ceramics, hydrogels, intraocular implants and transurethral systems.

64. The method of claim 54, 55, 56, 57 or 58 wherein the device is ethylenevinyl-acetate.

65. The method of claim 44, 45, 49, 52 or 53 wherein the chemotactic factor is selected from the group consisting of chemokines, nucleotides and neuropeptides.

66. The method of claim 46, 47, 48, 50, 51 or 65 wherein the chemokine is selected from the group consisting of MIP-1α, RANTES, MCP-3, MIP-5, MCPs, TARC, MDC, MIP-3α, IL-8, SDF-1, MIP-3β and SLC.

67. The method of claim 65 wherein the nucleotide is selected from the group consisting of ADP, UTP and UDP.

68. The method of claim 65 wherein the neuropeptide is selected from the group consisting of calcitonin-related gene protein and α-melanocyte-stimulating hormone.

69. The method of claim 46, 47, 48, 50, 51 or 65 wherein the chemokine is MIP-3β.

70. The method of claim 49, 50, 52 or 53 wherein the APC stimulating factor is selected from the group consisting of reactive haptens; cytokines, bacterial products, and ultraviolet radiation.

71. The method of claim 51 or 70 wherein the reactive hapten is selected from the group consisting of dinitroflourobenzene, fluorescein isothiocyanate, oxazolone and urushiol.

72. The method of claim 70 wherein the cytokine is selected from the group consisting of tumor necrosis factory and Interleukin 1.

73. The method of claim 70 wherein the bacterial product is selected from the group consisting of lipopolysacharrides and lipoproteins.

74. The method of claim 52 or 53 wherein the immunoregulatory molecule is selected from the group consisting of antigens, immunostimulatory molecules and immunosuppressive molecules.

75. The method of claim 74 wherein the antigen is selected from the group consisting of tumor-associated antigens, self antigens, allogeneic antigens, xenogeneic antigens and infectious disease-associated antigens.

76. The method of claim 75 wherein the tumor-associated antigen is selected from the group consisting of immunoglobulin idiotype, TCR, mutant p21/ras, mutant p53, p210/ber-abl fusion product, MART-1/melan A, MAGE-1, the GAGE family, human papilloma virus antigen, Epstein Bar virus antigen, tyrosinase, gp100, prostatic acid phosphatase, prostatic-specific antigen, prostate-specific membrane antigen, thyroglobulin, Her-2/neu, carcinoembryonic antigen, telomerase and Muc-1.

77. The method of claim 74 wherein the immunostimulatory molecule is selected from the group consisting of cytokines, co-stimulatory receptors, and bacterial products.

78. The method of claim 77 wherein the cytokine is selected from the group consisting of GM-CSF, interferon-α, interferon-β, interferon-γ, IL-1, IL-2, IL-6, IL-7, IL-12, IL-15 and TNFα.

79. The method of claim 77 wherein the co-stimulatory receptor is selected from the group consisting of CD40 ligand and inducible co-stimulatory protein.

80. The method of claim 77 wherein the bacterial product is selected from the group consisting of lipopolysacharrides and lipoproteins.

81. The method of claim 74 wherein the immunosuppressive molecule is selected from the group consisting of cytokines, soluble co-stimulatory molecules, neuropeptides, death ligands, and immunosuppressive chemicals.

82. The method of claim 81 wherein the cytokine is selected from the group consisting of IL-4, IL-10, IL-16 and transforming growth factor-β.

83. The method of claim 81 wherein the soluble co-stimulatory molecule is selected from the group consisting of cytotoxic T lymphocyte antigen 4-immunoglobulin, CD80-Ig, CD86-Ig and ICOS-Ig.

84. The method of claim 81 wherein the neuropeptide is selected from the group consisting of CGRP and α-MSH.

85. The method of claim 81 wherein the death ligand is selected from the group consisting of CD95 ligand and TRAIL.

86. The method of claim 81 wherein the immunosuppressive chemical is selected from the group consisting of corticosteroids, cyclosporin A, and FK506.

87. A vaccine comprising one or more chemotactic factor(s), one or more APC stimulating factor(s), and one or more immunoregulatory molecule(s).

88. The vaccine of claim 87 wherein the chemotactic factor is selected from the group consisting of chemokines, nucleotides and neuropeptides.

89. The vaccine of claim 88 wherein the chemokine is selected from the group consisting of MIP-1α, RANTES, MCP-3, MIP-5, MCPs, TARC, MDC, MIP-3α, IL-8, SDF-1, MIP-3β and SLC.

90. The vaccine of claim 88 wherein the nucleotide is selected from the group consisting of ADP, UTP and UDP.

91. The vaccine of claim 88 wherein the neuropeptide is selected from the group consisting of calcitonin-related gene protein and α-melanocyte-stimulating hormone.

92. The vaccine of claim 88 wherein the chemokine is MIP-3β

93. The vaccine of claim 87 wherein the APC stimulating factor is selected from the group consisting of reactive haptens, cytokines, bacterial products and ultraviolet radiation.

94. The vaccine of claim 93 wherein the reactive hapten is selected from the group consisting of dinitroflourobenzene, fluorescein isothiocyanate, oxazolone and urushiol.

95. The vaccine of claim 93 wherein the cytokine is selected from the group consisting of tumor necrosis factor-α and Interleukin 1.

96. The vaccine of claim 93 wherein the bacterial product is selected from the group consisting of lipopolysacharrides and lipoproteins.

97. The vaccine of claim 87 wherein the immunoregulatory molecule is selected from the group consisting of antigens, immunostimulatory molecules and immunosuppressive molecules.

98. The vaccine of claim 97 wherein the antigen is selected from the group consisting of self antigens, allogeneic antigens, xenogeneic antigens tumor-associated antigens and infectious disease-associated antigens.

99. The vaccine of claim 98 wherein the tumor-associated antigen is selected from the group consisting of immunoglobulin idiotype, TCR, mutant p21/ras, mutant p53, p210/ber-abl fusion product, MART-1/melan A, MAGE-1, the GAGE family, human papilloma virus antigen, Epstein Bar virus antigen, tyrosinase, gp100, prostatic acid phosphatase, prostatic-specific antigen, prostate-specific membrane antigen, thyroglobulin, Her-2/neu, carcinoembryonic antigen, telomerase and Muc-1.

100. The vaccine of claim97 wherein the immunostimulatory molecule is selected from the group consisting of cytokines, co-stimulatory receptors, and bacterial products.

101. The vaccine of claim 100 wherein the cytokine is selected from the group consisting of GM-CSF, interferon-α, interferon-β, interferon-γ, IL-1, IL-2, IL-6, IL-7, IL-12, IL-15 and TNFα.

102. The vaccine of claim 100 wherein the co-stimulatory receptor is selected from the group consisting of CD40 ligand and inducible co-stimulatory protein.

103. The vaccine of claim 100 wherein the bacterial product is selected from the group consisting of lipopolysacharrides and lipoproteins.

104. The vaccine of claim 97 wherein the immunosuppressive molecule is selected from the group consisting of cytokines, soluble co-stimulatory molecules, neuropeptides, death ligands, and immunosuppressive chemicals.

105. The vaccine of claim 104 wherein the cytokine is selected from the group consisting of IL-4, IL-10, IL16 and transforming growth factor-β.

106. The vaccine of claim 104 wherein the soluble co-stimulatory molecule is selected from the group consisting of cytotoxic T lymphocyte antigen 4-immunoglobulin, CD80-Ig, CD86-Ig and ICOS-Ig.

107. The vaccine of claim 104 wherein the neuropeptide is selected from the group consisting of CGRP and α-MSH.

108. The vaccine of claim 104 wherein the death ligand is selected from the group consisting of CD95 ligand and TRAIL.

109. The vaccine of claim 104 wherein the immunosuppressive chemical is selected from the group consisting of corticosteroids, cyclosporin A, and FK506.

110. The vaccine of claim 87 wherein the chemotactic factor is incorporated into a device.

111. The vaccine of claim 87 wherein the immunoregulatory molecule is incorporated into a device.

112. The vaccine of claim 87 wherein the chemotactic factor and the immunoregulatory molecule are incorporated into a device.

113. The vaccine of claims 110, 111 or 112 wherein the device is selected from the group consisting of is selected from the group consisting of nondegradable implant systems, biodegradable implant systems, implantable pump systems and atypical implantable pump systems.

114. The vaccine of claim 113 wherein the nondegradable implant system is selected from the group consisting of polymeric matrix systems (monolitic systems); reservoir systems; and bead systems, such as polymethylmethacrylate (PMMA) and polydimethylalloxane (PDMS) beads.

115. The vaccine of claim 113 wherein the biodegradable implant system is selected from the group consisting of reservoir systems; polymer monolitic systems, such as polyglycolic acid, polyactic acid, polyglycolic-lactic acid, polycaprolactone, ethylene-vinylacetate and actic acid/lysine; biodegradable copolymers with nondegradable coatings, such as ethylene-vinyl-acetate/methacrylate.

116. The vaccine of claim 113 wherein the implantable pump system is selected from the group consisting of infusion pumps, peristaltic pumps, osmotic pumps, positive displacement pumps and controlled release micropumps.

117. The vaccine of claim 113 wherein the atypical implantable pump system is selected from the group consisting of ceramic composites, inorganic bone meal or ossograft, alminium calcium phosphorous oxide ceramics, hydroxyapetite ceramics, tricalcium phosphate and amino acid antibiotic composite ceramics, hydrogels, intraocular implants and transurethral systems.

118. The vaccine of claim 113 wherein the device is ethylene-vinyl-acetate.

119. The vaccine of claim 87 wherein the loaded antigen presenting cells migrate to draining lymph nodes.

120. A method of regulating an immune response in a subject comprising administering to said subject the vaccine of claim 87 through 119.

121. The method of claim44, 45, 49, 52 or 53 wherein the chemotactic factor is administered subcutaneously.

122. The method of claim 49, 50, 52 or 53 wherein the APC stimulating factor is administered topically.

123. The method of claim 52 or 53 wherein the immunoregulatory molecule is administered subcutaneously.

124. The vaccine of claim 87 wherein the chemotactic factor is administered subcutaneously.

125. The vaccine of claim 87 wherein the APC stimulating factor is administered topically.

126. The vaccine of claim 87 wherein the immunoregulatory molecule is administered subcutaneously.

127. The composition of claim 32 wherein the self antigen is selected from the group consisting of Rh blood group antigens, platelet integrins, non-collangenous domain of basement membrane collagen type IV, epidermal cadherins, stroptococcal cell-wall antigens, rheumatoid factor IgG complexes, DNA, histones, ribosomes, snRNP, scRNP, pancreatic betacell antigen, unknown synovial joint antigen, myelin basic protein, protolipid protein, and myelin oligodendrocyte glycoprotein.

128. The composition of claim 32 wherein the allogeneic antigen is selected from the group consisting of class I major histocompatability complex molecules, class II major histocompatability complex molecules, non-classical major histocompatability complex molecules and minor antigens.

129. The method of claim 75 wherein the self antigen is selected from the group consisting of Rh blood group antigens, platelet integrins, non-collagenous domain of basement membrane collagen type IV, epidermal cadherins, stroptococcal cell-wall antigens, rheumatoid factor IgG complexes, DNA, histones, ribosomes, snRNP, scRNP, pancreatic betacell antigen, unknown synovial joint antigen, myelin basic protein, protolipid protein, and myelin oligodendrocyte glycoprotein.

130. The method of claim 75 wherein the allogeneic antigen is selected from the group consisting of class I major histocompatability complex molecules, class II major histocompatability complex molecules, non-classical major histocompatability complex molecules and minor antigens.

131. The vaccine of claim 98 wherein the self antigen is selected from the group consisting of Rh blood group antigens, platelet integrins, non-collagenous domain of basement membrane collagen type IV, epidermal cadherins, stroptococcal cell-wall antigens, rheumatoid factor IgG complexes, DNA, histones, ribosomes, snRNP, scRNP, pancreatic betacell antigen, unknown synovial joint antigen, myelin basic protein, protolipid protein, and myelin oligodendrocyte glycoprotein.

132. The vaccine of claim 98 wherein the allogeneic antigen is selected from the group consisting of class I major histocompatability complex molecules, class II major histocompatability complex molecules, non-classical major histocompatability complex molecules and minor antigens.
